(19) United States
(12) Patent Application Publication
(10) Pub. No.: US 2026/0251567 A1
(43) Pub. Date: Aug. 27, 2026

(54) SYSTEM FOR DETERMINING THE ATTENUATION OF A LIGHT WAVE PASSING THROUGH A SAMPLING VOLUME

(71) Applicants: CENTRE NATIONAL DE LA RECHERCHE SCIENTIFIQUE, Paris (FR); UNIVERSITE CLERMONT AUVERGNE, Clermont-Ferrand (FR)

(72) Inventors: Pierre COUTRIS, Volvic (FR); Guy FEBVRE, Aurillac (FR); Jean-François FOURNOL, Durtol (FR); William BOUCHER, Ceyrat (FR)

(73) Assignees: CENTRE NATIONAL DE LA RECHERCHE SCIENTIFIQUE, Paris (FR); UNIVERSITE CLERMONT AUVERGNE, Clermont-Ferrand (FR)

(21) Appl. No.: 18/879,395

(22) PCT Filed: Jun. 26, 2023
(86) PCT No.: PCT/EP2023/067308

§ 371 Date: Dec. 27, 2024

(30) Foreign Application Priority Data
Jun. 27, 2022 (FR) FR2206413

(51) Int. Cl. G01N 21/47; G01N 21/49; G01N 21/59

(52) U.S. Cl. CPC G01N 21/538; G01N 15/0211; G01N 15/06; G01N 15/075; G01N 2015/0046; G01N 2021/4711; G01N 33/1873

(57) ABSTRACT

A system for determining the attenuation of a light wave passing through a sampling volume, including a measurement part with an emission source emitting a collimated optical beam; a reference measurement channel measuring a reference optical intensity emitted by the emission source; a transmission measurement channel measuring a transmitted optical intensity in the sampling volume; a scattering measurement channel measuring a scattered optical intensity in the sampling volume; a guide assembly; and a control part controlling the measurement channels in order to carry out the transmission and scattering measurements concomitantly, and to determine the attenuation of the collimated optical beam in the sampling volume according to the reference optical intensity, the transmitted and scattered optical intensity, and the scattered optical intensity.

### TECHNICAL FIELD

The invention relates to a system for determining the attenuation of a light wave passing through a sampling volume, and to a method for determining the attenuation of a light wave passing through a sampling volume.

Clouds play a key role in the Earth's radiation balance, due to the strong interaction between the hydrometeors (water droplets and/or ice crystals) that make them up with solar and terrestrial radiation.

Improving our knowledge of cloud-radiation interactions and understanding the role of different clouds in the climate system requires a thorough understanding of their optical and microphysical properties, in order to implement cloud-representative properties in atmospheric models.

Extinction (or the resulting optical depth) is a fundamental optical property. It characterizes the attenuation of a light wave passing through a medium due to absorption and scattering processes. It is often described using the extinction coefficient (by volume), denoted p and defined as follows:

$$β ( λ ) = {c}_{abs} ( λ ) + {c}_{sca} ( λ )$$

Where λ is the wavelength of the incident wave, c_{abs} is the absorption coefficient (in m⁻¹), and c_{sca} is the scattering coefficient (in m⁻¹).

Within the context of the invention, in order to simplify the equations, it is assumed that c_{abs}=0 (extinction coefficient β(λ)=c_{sca}(λ)), and wavelength dependence is omitted (β(λ)=β), since the system is considered to be monochromatic.

The scattering coefficient is defined from the effective volume cross-section μ(θ, φ) of scattering by the following equation:

$${c}_{sca} = {\int }_{4 π} μ ( θ , φ ) sin ( θ ) d θ d φ$$

In this equation, θ∈[0−π] and φ∈[0−2π] are the polar and azimuth angles respectively.

When the scattering volume contains a population of particles of sizes D∈[Dₘᵢₙ−Dₘₐₓ] characterized by a dimensional distribution N(D), the effective volume cross-section of scattering is then given by:

$$μ ( θ , φ ) = {\int }_{{D}_{min}}^{{D}_{max}} ( {σ}_{sca} ( θ , φ , D ) ) * N ( D ) dD$$

Where N(D) dD is the concentration of particles of sizes between D and D+dD (in m⁻³), σ_{sca}(D) is the mean effective cross-sectional area of particles of sizes between D and D+dD (in m²).

Characterizing the optical properties of ice clouds with their complex microstructure, due to the great horizontal and vertical variability of the physical properties (size, shape, mass, presence of inclusions, surface roughness, effective scattering cross-section) of the hydrometeors that make them up, is one of the current scientific challenges, and on this point, in situ measurement, that is, carried out in the clouds themselves, presents a major challenge in this field.

In the atmosphere, optical extinction can be estimated in different ways. For example, using active remote sensing equipment such as the CALIOP lidar onboard the CALIPSO satellite, extinction can be deduced from backscattered power measurements, subject to strong assumptions about the multiple scattering coefficient and/or the lidar ratio, which depend on the type of target (aerosol, droplet, crystal). Furthermore, this technique only works through optically thin media wherein the attenuation of the laser beam is not too high.

Other instruments, such as optical spectrometers and polar nephelometers, can also be used on airborne measurement platforms. However, these instruments are not suitable for direct in-situ measurement. Extinction is deduced from measurements of granulometry or scattering indicators, under strong assumptions, which induce a high degree of uncertainty.

To measure extinction in a volume directly, we generally use a transmissiometer, a radiometric device that measures the attenuation of a light beam passing through a cloud volume. The principle is shown in FIG. 1. A light beam FL of incident intensity I₀ is emitted by an emission source SE, and collimated by a collimating lens LC. The beam passes through a sampling volume VE characterized by a volume extinction β and a depth L. The transmitted intensity I is collected by a focusing lens LF, then measured by a detector DE after passing through a pinhole ST.

Beam attenuation is described by the Beer-Bouguer-Lambert law:

$$I / {I}_{0} = exp ( - β L )$$

Where I and I₀ correspond respectively to incident and transmitted intensity (in W·m²), β corresponds to the extinction coefficient by volume (in m⁻¹), and L corresponds to the geometric length through the sampled medium (in m).

Determining the extinction coefficient by applying the Beer-Bouguer-Lambert law therefore presupposes the ability to measure the transmitted intensity, that is, the photons that have not interacted with the hydrometeors by scattering or absorption. It is therefore necessary to be able to differentiate the light actually transmitted without interacting with the particles from that which has been scattered forward, which is very difficult to achieve in practice.

Several types of optical arrangement have been proposed for this transmission measurement, but all have a non-zero field of view, characterized by the ratio between the diameter of the pinhole ST (non-zero) and the focal length of the lens LF (not infinitely large). The detector of this type of device therefore intercepts a fraction of the radiation scattered by the particles in an angular sector close to the front, that is, very close to the direction of propagation of the incident wave.

Since the detector intercepts a fraction of the energy scattered by the particles, this is added to the transmitted energy, and distorts the transmission measurement. The extinction coefficient deduced from the uncorrected transmission measurement is therefore systematically underestimated. The error committed, intrinsic to the measurement principle of transmissiometers, depends on the optical characteristics of the transmission measurement device (its field of view), and on the optical properties of the observed particle population (effective volume cross section or scattering coefficient and phase function). It is particularly important in the case of objects that scatter light very strongly towards the front, such as the crystals that make up ice clouds.

The paper “An Instrument For The Measurement Of Spectral Attenuation Coefficient And Narrow Angle Volume Scattering Function Of Ocean Waters” (Austin, R. W. and T. J. Petzold, Proc. SPIE 0064, Ocean Optics IV, Nov. 10, 1975) presents a submersible instrument for studying optical attenuation in the ocean. The instrument performs a transmission measurement in the sampling volume, followed by three measurements of the effective volume cross-section at three polar angles close to the optical axis (4, 8 and 16 mrad, with 0 mrad indicating the direction of light propagation)

Austin and Petzold's paper describes the device used to successively perform these four measurements, using a wheel mechanism comprising a circular aperture (pinhole for the transmission measurement) and three annular apertures of different sizes defining the fields of view for the three scattering measurements.

However, the instrument presented in this article has the disadvantage of performing transmission and scattering measurements sequentially. When applying this instrument to the characterization of hydrometeors present in a moving cloud, sequential measurements would distort the measurement. This is because the sampling volume would not be identical from one instant of measurement to the next.

Another drawback of the prototype presented in the article is that the angles at which scattering is measured are limited in number (three in the article) and fixed, as they are mechanically determined by the size of the annular openings. They cannot therefore be modified by the user during measurement and/or post-processing.

Moreover, in Austin and Petzold's paper, the effective volume cross-section is not resolved according to azimuthal angle (p: the use of an annular mask and a single-element detector produces a measurement of scattered intensity integrated between 0 and 2π.

In addition, the instrument is particularly well suited to aqueous media, where attenuation is much higher than in the atmosphere, so measurements can be conclusive even with a sampling volume length of the order of one or two meters between the emission source and the receiving channels. Thus, in the article a spacer is used to maintain alignment between the emission and receiving parts.

In the case of less attenuating media, such as most clouds in the earth's atmosphere, it would be necessary to greatly increase the length between the emission source and the receiving channels. A spacer several meters long, sufficiently rigid to maintain optical alignment over such a length, would be difficult to envisage, and would require precise alignment mechanisms, increasing the complexity and cost of the system.

For optical extinction measurements in the atmosphere, it would be necessary to greatly increase the length between the emission source and the receiving channels. As a spacer of several meters is difficult to envisage, precise alignment mechanisms would be required, increasing the complexity and cost of the system.

There is therefore a need for systems and methods for determining the attenuation of a light wave passing through a sampling volume, enabling transmission and scattering measurements close to the optical axis to be carried out coincidentally and co-locally for all types of media, and for mapping the effective volume cross-section over a wide range of polar and azimuthal angles with sufficient angular resolution in both dimensions (polar and azimuthal).

### SUMMARY OF THE INVENTION

An object of the invention is therefore a system for determining the attenuation of a light wave passing through a sampling volume, comprising:
a measurement part comprising:
   an emission source emitting a collimated optical beam;
   a reference measurement channel, configured to measure a reference optical intensity emitted by the emission source;
   a transmission measurement channel, configured to measure a transmitted optical intensity in the sampling volume and scattered in the sampling volume in a polar angle range between 0° and a collection angle θ_{coll}>0, the polar angle being defined with respect to the optical axis of the collimated optical beam, where 0° indicates the direction of propagation;
   a scattering measurement channel, configured to measure a scattered optical intensity in the sampling volume, at a plurality of polar angles with respect to the optical axis of the collimated optical beam between θ_{coll} and a maximum detection angle θₘₐₓ>θ_{col}, in order to characterize the properties of the sampling volume in a near forward angular sector;
   a guide assembly, configured to guide the collimated optical beam from the emission source to the measurement channels through the sampling volume;
   a control part, configured to control the measurement channels in order to carry out the transmission and scattering measurements concomitantly, and to determine the attenuation of the collimated optical beam in the sampling volume according to the reference optical intensity, the transmitted and scattered optical intensity, and the scattered optical intensity.

Advantageously, the guide assembly comprises a plurality of beam splitters for transmitting the collimated optical beam to the reference measurement channel, to the transmission measurement channel and to the scattering measurement channel, and at least one optical retroreflector, arranged along the optical axis of the emission source, and configured to fold back on itself the optical beam having passed through the sampling volume in the direction of the emission source.

Advantageously, the scattering measurement channel comprises a scattering objective lens and a multi-element optoelectronic sensor configured to image the Fourier plane of the scattering objective lens.

Advantageously, an absorbing element, configured to absorb the optical beam transmitted and scattered between 0° and θ_{coll}, covers the pixels of the central part of the multi-element optoelectronic sensor.

Advantageously, the absorbing element has an absorption rate that decreases from the central part of the multi-element optoelectronic sensor towards the ends of the multi-element optoelectronic sensor.

Advantageously, the multi-element optoelectronic sensor has no optical acquisition elements in its central part.

Advantageously, the multi-element optoelectronic sensor has a sufficiently high dynamic range to measure the transmitted intensity and to measure the scattered intensity.

Advantageously, the multi-element optoelectronic sensor is a CMOS sensor.

Advantageously, the scattering measurement channel comprises an occulter arranged in the focal plane of the scattering objective lens and an assembly of at least two lenses, configured to image the Fourier plane of the scattering objective lens onto the multi-element optoelectronic sensor through a Lyot diaphragm.

Advantageously, the transmission measurement channel comprises a transmission objective lens, and a pinhole of radius rₜ placed at focal distance fₜ of the transmission objective lens, where tan(θ_{coll})=rₜ/fₜ.

Advantageously, the control part is configured to:
determine the scattering effective volume cross-sections of the sampling volume from the optical intensity scattered in the sampling volume at different angles to the optical axis of the collimated optical beam;
apply a curve adjustment to the scattering effective volume cross-section values, and extrapolate the curve obtained to the scattering effective volume cross-section values between 0° and θ_{coll};
determine the extinction coefficient deduced from the transmission measurement contaminated by forward scattering β* with the following relationship:

$${β}^{*} = β - {\int }_{0}^{2 π} {\int }_{0}^{{θ}_{coll}} μ ( θ , φ ) sin ( θ ) d θ d φ$$

Where β corresponds to the real extinction coefficient calculated by the Beer-Bouguer-Lambert law, μ(θ, φ) corresponds to the value of effective volume cross-sections that varies as a function of the polar angle θ and the azimuthal angle of rotation around the optical axis φ

Advantageously, the emission source, the reference measurement channel, the transmission measurement channel and the scattering measurement channel are integrated into a single housing.

The invention also relates to a method for determining the attenuation of a light wave passing through a sampling volume, comprising at least one iteration consisting in carrying out:
emitting a collimated optical beam by an emission source;
measuring a reference optical intensity emitted by the emission source;
measuring an optical intensity transmitted into the sampling volume and scattered in the sampling volume in a polar angle range between 0° and a collection angle θ_{coll}>0, the polar angle being defined relative to the optical axis of the collimated optical beam, where 0° indicates the direction of propagation;
measuring an optical intensity scattered in the sampling volume, at a plurality of polar angles to the optical axis of the collimated optical beam between θ_{coll} and a maximum detection angle θₘₐₓ>θ_{coll}, in order to characterize the properties of the sampling volume in a near forward angular sector;
checking the measurements carried out concomitantly, and a determination of the attenuation as a function of the reference optical intensity, the transmitted optical intensity and the scattered optical intensity.

Advantageously, the method comprises a plurality of iterations.

Advantageously, the sampling volume is a cloudy environment, in particular an ice-phase cloud.

### DESCRIPTION OF FIGURES

Further features, details and advantages of the invention will become apparent upon reading the description made with reference to the appended drawings given by way of example.

FIG. 1, already described, shows the principle of transmission measurement, from which we then deduce extinction by applying the Beer-Bouguer-Lambert law.

FIG. 2 shows the architecture of the system according to the invention.

FIG. 3 is a schematic diagram of the measurement part of the system.

FIG. 4 shows the transmission measurement channel.

FIG. 5 shows one embodiment of the scattering measurement channel.

FIG. 6 shows a front view of an absorbent element according to the invention.

FIG. 7 shows an embodiment of the scattering measurement channel according to one embodiment of the invention.

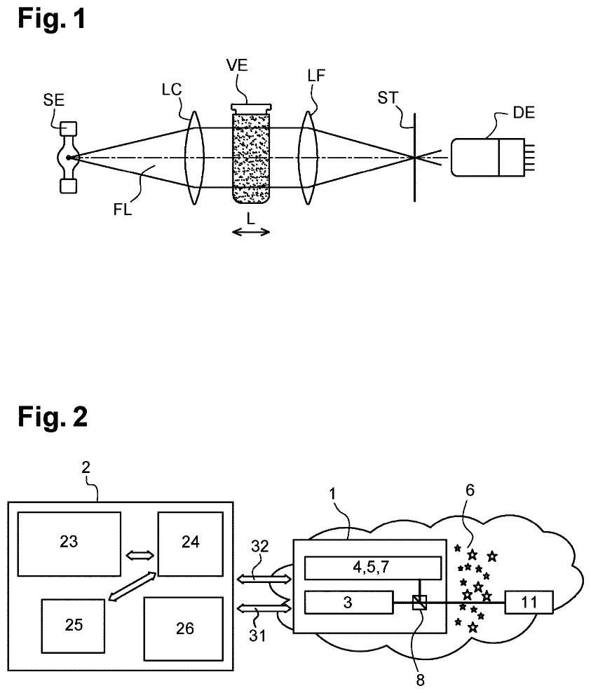

The invention is described with reference to FIG. 2, which shows the system very schematically.

The system comprises a measurement part 1 comprising the emission and detection modules required for measurement, and a control part 2 via which an operator controls the instrument and data acquisition.

The control part 2 comprises in particular a power supply sub-part 26 for supplying the measurement part 1 and the control part 2 with electrical power. An electrical link 31 connects the power supply sub-part 26 to the measurement part 1.

The control part 2 also comprises a control and acquisition sub-part 24 for gathering all the parameters involved in the measurement and supplying them to the measurement part 1 via an external computer link 32. The control and acquisition sub-part 24 is also responsible for controlling the elements of the measurement part 1, collecting their status and managing the system's operating modes. It can also acquire service and monitoring information (e.g., operating temperatures or supply voltages), and manage alarms with immediate reaction and/or information.

Measurement and environmental parameters are stored in a storage sub-part 25, which is computer-linked to the control, command and acquisition sub-part 24.

Parameters are saved in a format that can be exported and used with computer hardware and software.

A man-machine interface sub-part 23, computer-linked to the control, command and acquisition sub-part 24, enables a user to interact with the various measurement elements, and to view or preview the measurement results for further processing. The data displayed can be, for example, graphs representing the time series of transmitted and received power, or the attenuation measurement. The man-machine interface sub-part 23 can also enable the user to start and stop measurement, control the modification of measurement parameters, or display the status of system modules, measurement and environmental parameters, and alarms and various safety information.

The measurement part 1 comprises an emission source 3 for a collimated optical beam, a reference measuring channel 4, a transmission measuring channel 5, a scattering measuring channel 7, and a guide assembly for guiding the collimated optical beam from the emission source 3 to the measuring channels (4, 5, 7) through the sampling volume 6. Sampling volume 6 may be, for example, an atmospheric sampling volume, or an aquatic sampling volume, or any other medium wherein an optical extinction measurement is to be carried out.

The measurement part 1 is exposed to the sampling volume 6, and the control part 2 can be either partially or totally exposed to the sampling volume 6, or located outside the sampling volume 6 for remote control.

In one embodiment of the invention, the measurement part 1 can be located outside a building, for example on the roof of the building, and the control part 2 inside the building. In another embodiment, the measurement part 1 can be located partly outside an aircraft (on the fuselage and/or under a wing), and the control part 2 can be housed in the aircraft cabin.

The control part 2 controls the reference measurement channel 4, the transmission measurement channel 5 and the scattering measurement channel 7 in order to carry out transmission and scattering measurements concomitantly, and to determine the attenuation of the collimated optical beam in the sampling volume 6 according to the reference optical intensity I₀ measured by the reference measurement channel 4, the transmitted and scattered optical intensity I_{T} measured by the transmission measurement channel 5, and the scattered optical intensity I_{D} measured by the scattering measurement channel 7. Since all scattering and transmission measurements are carried out concomitantly and co-located, it is possible to use the effective volume cross-section values measured by the scattering channel to correct the value of the transmitted and scattered optical intensity I_{T} measured by the transmission channel 5 before applying the Beer-Bouguer-Lambert formula to calculate extinction.

Indeed, the concomitance of transmission and scattering measurements, and the colocalization of their sampled volumes, makes it possible to estimate by calculation the proportion of scattered energy in what is measured by the transmission measurement channel 5, which improves the accuracy of determining the extinction coefficient compared with prior art solutions.

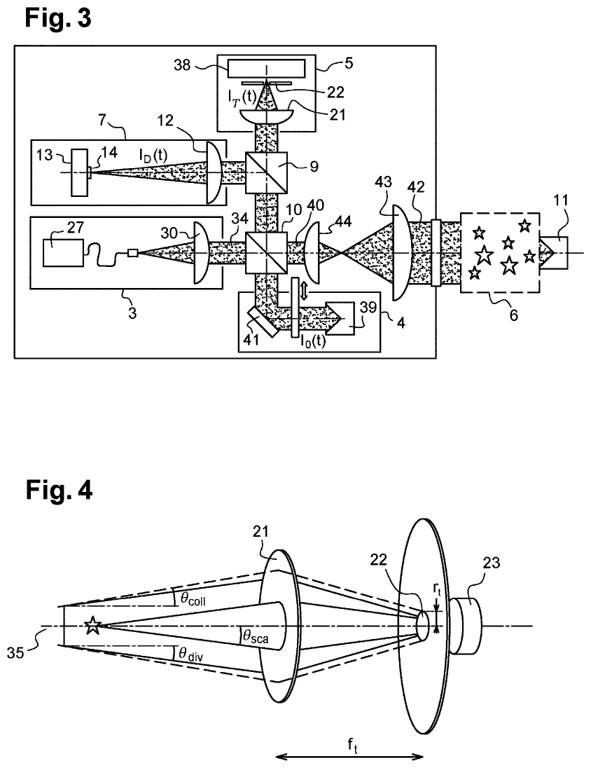

FIG. 3 shows the measurement part 1 in greater detail. The emission source 3 comprises an optoelectronic component 27 capable of emitting monochromatic light, characterized by a wavelength and an optical power. For example, optoelectronic component 27 may be a laser diode with wavelength λ=808 nm and power P≥1 W. The optical beam, after expansion, may have a diameter of around 45 mm, the diameter having to be compatible with the diameters of the optical elements in the guide assembly.

The characteristics of the emission source 3 (wavelength and optical power) can be adapted to the properties of the scattering particles to be characterized (size and concentration for example), just as the use of a polychromatic source, bandpass filters and/or polychromatic sensors make it possible to measure extinction at different wavelengths in media where this is relevant.

If a laser is used as an emission source, an isolator can be fitted to prevent source instability caused by light returning to the source.

The emission source 3 also includes a collimating lens 33, an iris to limit beam width, and possibly an internal baffling system.

A first beam splitter 10 separates the beam emitted by the emission source 3 into a beam directed towards the reference measurement channel 4 and a beam directed towards the sampling volume 6. Reference measurement channel 4 measures the amount of energy I₀ emitted by the emission source illuminating sampling volume 6. Measuring energy quantity I₀ can be measured by a component capable of capturing radiation from the optical domain and transforming it into an electrical signal, such as a photodiode. Alternatively, as shown in FIG. 3, measuring the energy quantity I₀ can be carried out by the transmission channel detector, such as a photodiode 38. In this case, the reference measurement channel 4 comprises a reflector 39 that sends the optical beam back to the transmission measurement channel 5, and a shutter 40 that selects the flux sent to the photodiode 38, which is either the reference flux or the flux that has interacted with the sampling volume. The selection can be made by translation of the shutter 40, or by any other means enabling such a selection to be made. For convenience, the beam of the reference measurement channel 4 can be folded using a flat mirror 41.

The ratio of the beam splitter 10 can be, for example, 50/50. This value of the reflection/transmission ratio optimizes the scattered energy arriving at the detector in a set-up where the beam is folded using the cube corner.

The width of the collimated optical beam 34 emanating from the source is widened by a doublet of lenses 43, 44, then passes through the sampling volume 6, represented in FIG. 3 by a cloudy environment made up of any hydrometeors (e.g., liquid water droplets, ice crystals), represented in the form of stars. The optical beam interacts with the ice crystals, creating a phenomenon of forward light scattering, that is, in the direction of optical flow transmission. A porthole 42 provides the interface between the inside of the housing and the outside environment.

The optical flow is reflected by the beam splitter 10, towards a second beam splitter 9, which separates the beam into two parts. The ratio of the second beam splitter 10 can be 90/10 (90% of the beam power to the scattering channel and only 10% to the transmission channel). In fact, when extinction is between 0.1 and 100 km⁻¹, the transmitted power is much greater than the scattered power (several orders of magnitude). Given the power of the source, there is more energy than necessary to carry out the transmission measurement, so the ratio of the second beam splitter 10 allows as much power as possible to be sent to the scattering channel.

An imaging relay (not shown in the figures) can be inserted between the two beam splitters, to image retroreflector 11 on the detection planes (transmission and scattering).

Part of it is transmitted on transmission measurement channel 5. In this way, the amount of energy I_{T} transmitted through the sampling volume 6 of thickness L and reaching a detector is compared with the incident energy I₀. The detector 38 is a component capable of capturing radiation from the optical domain and transforming it into an electrical signal, for example a photodiode. In practice, the same photodiode can be used to measure I₀ and I_{T}.

Transmitted energy I_{T} is measured using an optical device whose principle is shown in FIG. 4. The collimated incident beam (with low divergence, characterized by the half-angle θ_{div}) passes through the sampling volume and is then collected by a transmission objective lens 21 after having passed through the sampling volume and focused towards a detector, which can be a photodiode, through a pinhole-type device 22 (or needle hole) placed in the focal plane of the transmission objective lens 21.

In this case, the field of view of the transmission detector is a half-angle cone at the apex denoted θ_{coll} (generally slightly greater than θ_{div}) whose value is determined by the radius of the pinhole 22, denoted r, and by the focal length of the transmission objective lens 21, denoted fₜ, by the relation tan

$${θ}_{coll} = \frac{{r}_{t}}{{f}_{t}}$$

In this way, detector 23 is able to capture scattering objects from the sampled medium as well as scattered rays in a polar angle range between 0° and θ_{coll}, the polar angle being defined relative to optical axis 35 of collimated optical beam 34, where 0° indicates the direction of propagation, and θ_{coll} corresponds to the collection angle of the transmission measurement (typically of the order of a milliradian).

As can be seen in FIGS. 3 and 4, some of the light scattered forward by the particles making up the sampling volume 6 is collected by the detector 23 of the transmission measurement channel 5, and directly pollutes the transmission measurement, with a scattering angle θ_{sca} of between 0° and θ_{coll} relative to the optical axis of the optical beam.

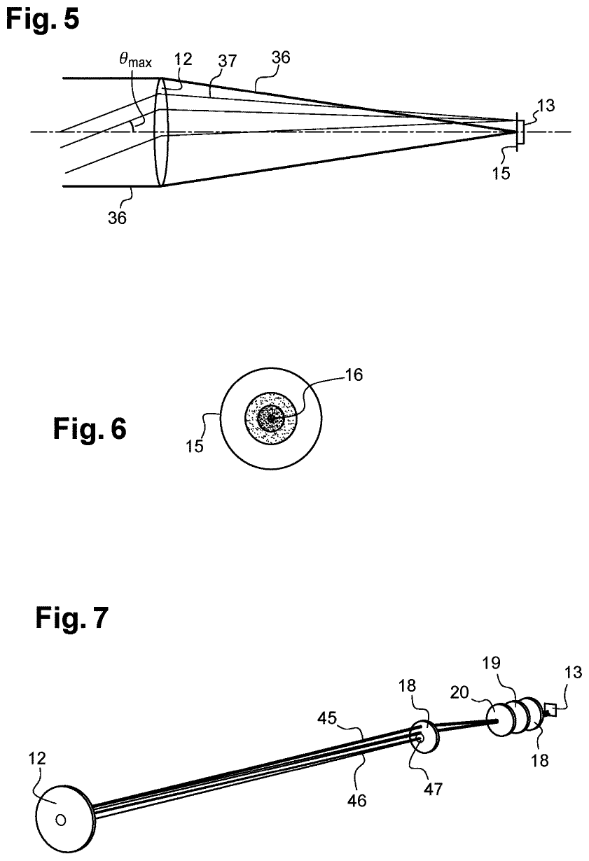

In order to overcome the problem of pollution of the transmission measurement, which collects part of the intensity scattered by the particles making up the sampling volume 6 in addition to the transmitted intensity, the system according to the invention comprises a scattering measurement channel 7, configured to measure a mapping of the effective volume scattering cross-section over a range of azimuthal angles between 0 and 2π, and polar angles with respect to the optical axis of the collimated optical beam 34 between θ_{coll} and θₘₐₓ, where θₘₐₓ corresponds to a maximum detection angle for characterizing the properties of the sampling volume 6 in a near-front angular sector (or HFOV for “Half Field Of View” of the detection system). Thus, θₘₐₓ is defined by the size of the multi-element optoelectronic sensor 13 and by the focal length of the scattering objective lens 12 (see FIG. 5).

This mapping, obtained with very fine angular resolution, makes it possible to define a correction function for the transmission measurement based on the quantities of scattered energy measured in different angular sectors, and to characterize many of the properties of the hydrometeors sampled (thermodynamic state, preferred orientations, size and concentration).

Adapted to the optical properties of hydrometeors, and easily adaptable to all two-phase flows wherein scattering of the light beam by suspended particles is not negligible, this solution reduces uncertainty in the determination of the extinction coefficient obtained from the transmission measurement.

According to an advantageous embodiment shown in FIGS. 3 and 4, an optical reflector 11 is arranged along the optical axis of the emission source 3. It folds the optical beam 34 back on itself that is passing through the sampling volume 6 towards the emission source 3.

The use of an optical retroreflector (“cube-corner” reflector) firstly enables the collimated optical beam to be folded, thus reducing the overall dimensions for a given optical length. It is understood that the Beer-Bouguer-Lambert law takes into account the total length of the optical path in sampling volume 6, in both directions of beam transmission.

This also enables the emission source 3, reference measurement channel 4, transmission measurement channel 5 and scattering measurement channel 7 to be integrated into a single housing, making it easier to maintain optical alignment of the various measurement modules with the source during measurements, as well as system installation and maintenance.

In addition, the use of an optical retroreflector preserves optical alignment between the emission source 3 and the transmission measurement channel 5 and scattering measurement channel 7, despite the distance separating them and the relative movements that may exist between the housing and the reflector. For extinguishing measurements in an atmospheric environment, a distance of several meters (between five and ten meters) is generally required to obtain conclusive measurements. This distance is created by folding the beam using a retroreflector.

State-of-the-art solutions require a sufficiently rigid mechanical structure over a length of several meters, or a servo mechanism, to maintain perfect alignment between the emission source and the measurement channels. This is all the more so for airborne applications, given the vibrations that can occur in the fuselage. Since, by optical construction, the rays are always reflected back towards their source (due to the three orthogonal reflecting planes that make up the retroreflector), the use of a retroreflector simplifies installation and makes the device tolerant to retroreflector misalignment, while dispensing with the need for a servo mechanism.

However, the presence of a retroreflector is not essential to the implementation of the system according to the invention. For example, for extinction measurements in media where the measurement can be carried out with a sampling volume length of a few tens of centimeters, or for measurements carried out in the laboratory, in a very stable environment, it may be envisaged that the emission source and the measurement channels are positioned opposite one another. In this case, they would be separated by the sampling volume.

According to one embodiment, the scattering measurement channel 7 comprises a scattering objective lens 12 and a multi-element optoelectronic sensor 13 configured to image the Fourier plane of the scattering objective lens 12. In addition, the scattering measurement channel must enable the effective volume cross-section to be mapped from the intensity of the light scattered in the sampling volume at polar angles between θ_{coll} and θₘₐₓ, bearing in mind that the power transmitted and scattered between 0° and θ_{coll} also arrives on this measurement channel and may be very much greater than the scattered power values to be measured.

As can be seen in FIGS. 3 and 5, the part 36 of the optical beam that was transmitted to the detector 23 of the transmission measurement channel 5 is not picked up by the multi-element optoelectronic sensor 13.

Placing the optoelectronic multi-element sensor 13 at the focal distance f_{d} from the objective scattering lens 12 and keeping the latter perfectly aligned with the transmitted beam makes it possible to obtain a correspondence between the direction wherein light is scattered in the sampling volume, characterized by the angles θ and φ, and the radial position of the pixel of the multi-element optoelectronic sensor 13 that measures this scattered light (distance r_{d} from the point on the multi-element optoelectronic sensor 13 that coincides with the optical axis), with the relationship: r_{d}=f_{d}·tan(θ).

The use of a multi-element sensor enables angularly resolved measurement along φ, unlike the ring system of Austin and Petzold 1975, which achieves integrated measurement along [0−2π]. This is yet another advantage of using a multi-element sensor. Measurements indicate in which media this property is satisfied, and if not, allow us to deduce information about crystal orientation and shape.

In this way, the invention makes it possible to obtain, in a single shot, a fine angular resolution mapping of the light power scattered over a range of polar angles of interest between θ_{coll} and θₘₐₓ and azimuthal angles between 0 and 2π, by the particles that attenuate the beam, at the same time as the transmission measurement. The invention overcomes the problem of intra- and inter-cloud variability in the constituent parameters of hydrometeors (size, shape, surface roughness, etc.), which renders useless any attempt to correct the transmission measurement using theoretical and/or empirically determined correction functions based on laboratory measurements on artificial samples.

The multi-element optoelectronic sensor 13 can be a CMOS (Complementary Metal-Oxide-Semiconductor) sensor. CMOS sensors have a higher operating speed (frame rate) than other multi-element optoelectronic sensors, which is advantageous for scientific measurement applications. However, other optoelectronic sensors, such as CCD (Charge Coupled Device) sensors, could also be used to implement the invention. The angular resolution of the scattering measurement channel 7 depends on the resolution of the multi-element optoelectronic sensor 13. For a CMOS sensor, angular resolution well below one milliradian can be envisaged.

Unless you have a multi-element optoelectronic sensor 13 with sufficiently high dynamic range to measure transmitted intensity and to measure scattered intensity at certain polar angles to the optical axis of the collimated optical beam 34 between θ_{coll} and θₘₐₓ, or to be devoid of pixels in the central part corresponding to polar angles with respect to the optical axis of the collimated optical beam 34 between 0° and θ_{coll}, it is necessary to add an anti-glare device 14 to the multi-element optoelectronic sensor 13.

An anti-glare device 14 prevents the multi-element optoelectronic sensor 13 from being dazzled by the optical beam focused at its center, while enabling measurement of the optical intensity scattered in the sampling volume. In fact, the power ratio between the broadcast signal and the transmission signal can be greater than 1:10⁻⁶. Preferably, the anti-glare device 14 has the same dimensions as the pinhole 22, in the plane perpendicular to the optical axis 35.

According to a first embodiment, shown in FIG. 6, the anti-glare device 14 comprises an absorbent element 15 which is bonded to the protective pane of the multi-element optoelectronic sensor 13. The absorbent element 15, which may be in the form of a film, occludes the pixels of the central part 16 of the multi-element optoelectronic sensor, in order to absorb the optical beam transmitted and scattered between 0° and θ_{coll}. “AcktarBlack” (registered trademark), an absorbent film from “Acktar” (registered trademark), has very low reflectance, and is therefore suitable for this application.

The absorbent element 15 can have an absorption rate that decreases from the central part 16 of the multi-element optoelectronic sensor 13 towards the ends of the sensor, according to a predefined gradient, as shown in FIG. 6. This smooths the intensity to be measured over the sensor surface.

According to a variant shown by FIG. 7, the scattering measurement channel 7 comprises an occulter 47, placed just in front of the lens 18, arranged in the focal plane (Fourier plane) of the scattering objective lens 12, and an optical system consisting of at least two lenses (18, 19), configured to image this plane onto the multi-element optoelectronic sensor 13, possibly through a Lyot diaphragm 20. The imaging relay provided by lenses 18 and 19 enables the light-blocking device 17 to be placed in the Fourier plane, and this plane to be imaged using the multi-element optoelectronic sensor 13, which is physically positioned outside the Fourier plane, and therefore at a distance from the occulting disk. This provides a degree of freedom with regard to the positioning of the multi-element optoelectronic sensor 13. The incident beam 45 strikes the lens 18 at an angle less than θₘₐₓ and propagates through the device to the multi-element optoelectronic sensor 13. The incident beam 46, which corresponds to the transmitted beam scattered at angles below θ_{coll}, is blocked by the occulter 47.

A bandpass filter can be used in a monochromatic solution to ensure that only light from the source reaches the detector (for example, to filter out sunlight in an in situ measurement).

This variant prevents diffraction at the edges of the light-blocking device 17 and the objective lens 12 from contaminating the measurement of scattered intensity by the multi-element optoelectronic sensor 13.

The measurement of scattered power over a plurality of polar angles with respect to the optical axis of the collimated optical beam enables us to characterize effective the volume cross-sections of scattering of the medium over these same angles.

From the scattered optical intensity I_{D} in the sampling volume at different angles to the optical axis of the collimated optical beam 34, and the reference optical intensity I₀, the effective cross-sectional area values of the sampling volume 6 are determined by a linear law known to the person skilled in the art. A curve adjustment corresponding to the values of the effective volume cross-sections of scattering (e.g., interpolation of the values), then extrapolation of the curve obtained by adjusting towards the angle of transmission collection, enables us to estimate the effective volume cross-section of scattering between 0° and the angle θ_{coll}.

It is then possible to determine the extinction coefficient deduced from the transmission measurement contaminated by forward scattering β* with the following relationship:

$${β}^{*} = β - {\int }_{0}^{2 π} {\int }_{0}^{{θ}_{coll}} μ ( θ , φ ) sin ( θ ) d θ d φ$$

Where β corresponds to the real extinction coefficient calculated by the Beer-Bouguer-Lambert law, μ(θ, φ) corresponds to the value of effective volume cross-sections that varies as a function of the polar angle θ and the azimuthal angle of rotation around the optical axis φ

The invention also relates to the method implemented by the aforementioned system. The method can be carried out as a one-off operation, or can comprise a plurality of iterations over time, so as to follow a temporal evolution of the measured quantities.

The method is particularly suitable for characterizing a cloudy environment, in particular an ice-phase cloud. Indeed, ice-phase clouds are a typical example of a two-phase medium made up of strongly scattering particles of sizes well above visible wavelengths, for which light scattering, particularly forward scattering, cannot be neglected.

The method also has industrial applications, for example in two-phase flow characterization, granulometric analysis and visibility measurements in aeronautics.

1-15. (canceled)

16. A system for determining the attenuation of a light wave passing through a sampling volume, comprising:
a measurement part comprising:
an emission source emitting a collimated optical beam;
a reference measurement channel, configured to measure a reference optical intensity emitted by the emission source;
a transmission measurement channel, configured to measure a transmitted optical intensity in the sampling volume and scattered in the sampling volume in a polar angle range between 0° and a collection angle θ_{coll}>0, the polar angle being defined with respect to the optical axis of the collimated optical beam, where 0° indicates the direction of propagation;
a scattering measurement channel, configured to measure a scattered optical intensity in the sampling volume, at a plurality of polar angles relative to the optical axis of the collimated optical beam between θ_{coll} and a maximum detection angle θₘₐₓ>θ_{coll}, in order to characterize the properties of the sampling volume in a near forward angular sector;
a guide assembly, configured to guide the collimated optical beam from the emission source to the measurement channels through the sampling volume;
a control part, configured to control the measurement channels in order to carry out transmission and scattering measurements concomitantly, and to determine the attenuation of the collimated optical beam in the sampling volume according to the reference optical intensity, the transmitted and scattered optical intensity, and the scattered optical intensities.

17. The measurement system according to claim 16, wherein the guide assembly comprises a plurality of beam splitters for transmitting the collimated optical beam to the reference measurement channel, to the transmission measurement channel and to the scattering measurement channel, and at least one optical retroreflector, arranged along the optical axis of the emission source, and configured to fold the optical beam back on itself that has passed through the sampling volume in the direction of the emission source.

18. The measurement system according to claim 16, wherein the scattering measurement channel comprises a scattering objective lens and a multi-element optoelectronic sensor configured to image the Fourier plane of the scattering objective lens.

19. The measurement system according to claim 18, wherein an absorbing element, configured to absorb the optical beam transmitted and scattered between 0° and θ_{coll}, covers the pixels of the central part of the multi-element optoelectronic sensor.

20. The measurement system according to claim 19, wherein the absorbing element has an absorption rate that decreases from the central part of the multi-element optoelectronic sensor towards the ends of the multi-element optoelectronic sensor.

21. The measurement system according to claim 18, wherein the multi-element optoelectronic sensor is devoid of optical acquisition elements in its central part.

22. The measurement system according to claim 18, wherein the multi-element optoelectronic sensor has a dynamic range such that it can measure the transmitted and scattered intensity with a power ratio between the scattering signal and the transmission signal greater than 10⁻⁶.

23. The measurement system according to claim 18, wherein the multi-element optoelectronic sensor is a CMOS sensor.

24. The measuring system according to claim 18, wherein the scattering measurement channel comprises a light-blocking device arranged in the focal plane of the scattering objective lens and an assembly of at least two lenses, configured to image the Fourier plane of the scattering objective lens onto the multi-element optoelectronic sensor through a Lyot diaphragm.

25. The measuring system according to claim 16, wherein the transmission measurement channel comprises a transmission objective lens, and a pinhole of radius rₜ placed at focal distance fₜ of the transmission objective lens, where tan(θ_{coll})=rₜ/fₜ.

26. The measurement system according to claim 16, wherein the control part is configured to:
determine the effective volume scattering cross-sections of the sampling volume from the optical intensity scattered in the sampling volume at different angles to the optical axis of the collimated optical beam;
apply a curve adjustment to the scattering effective volume cross-section values, and extrapolate the curve obtained to the scattering effective volume cross-section values between 0° and θ_{coll};
determine the extinction coefficient deduced from the transmission measurement contaminated by scattering before β* with the following relationship:
$${β}^{*} = β - {\int }_{0}^{2 π} {\int }_{0}^{{θ}_{coll}} μ ( θ , φ ) sin ( θ ) d θ d φ$$
Where β corresponds to the real extinction coefficient calculated by the Beer-Bouguer-Lambert law, μ(θ, φ) corresponds to the value of effective volume cross-sections that varies as a function of the polar angle θ and the azimuthal angle of rotation around the optical axis φ.

27. The measurement system according to claim 16, wherein the emission source, the reference measurement channel, the transmission measurement channel and the scattering measurement channel are integrated in a single housing.

28. A method of determining the attenuation of a light wave passing through a sampling volume, comprising at least one iteration consisting in carrying out:
emission of a collimated optical beam by an emission source;
a measurement of a reference optical intensity emitted by the emission source;
a measurement of an optical intensity transmitted into the sampling volume and scattered in the sampling volume in a polar angle range between 0° and a collection angle θ_{coll}>0, the polar angle being defined relative to the optical axis of the collimated optical beam, where 0° indicates the direction of propagation;
a measurement of an optical intensity scattered in the sampling volume, at a plurality of polar angles to the optical axis of the collimated optical beam between θ_{coll} and a maximum detection angle θₘₐₓ>θ_{coll}, in order to characterize the properties of the sampling volume in a near forward angular sector;
a check of the measurements carried out concomitantly, and a determination of the attenuation as a function of the reference optical intensity, the transmitted optical intensity, and the scattered optical intensities.

29. The method according to claim 28, comprising a plurality of iterations.

30. The method according to one of claim 28, wherein the sampling volume is a cloudy environment, in particular an ice-phase cloud.
